(19) United States
(12) Patent Application Publication
(10) Pub. No.: US 2025/0122293 A1
(43) Pub. Date: Apr. 17, 2025

(54) CSF3R AS BIOMARKER AND THERAPEUTIC TARGET FOR PULMONARY FIBROSIS

(71) Applicant: FNCT BIOTECH, INC., Seoul (KR)

(72) Inventors: Su Jae LEE, Seoul (KR); Jae Hyeok KANG, Seoul (KR); Da In LEE, Seoul (KR); Eun Ji LIM, Seoul (KR); In Yeong YUN, Seoul (KR)

(73) Assignee: FNCT BIOTECH, INC., Seoul (KR)

(21) Appl. No.: 18/689,582

(22) PCT Filed: Sep. 7, 2022
(86) PCT No.: PCT/KR2022/013410

§ 371 Date: Mar. 6, 2024

(30) Foreign Application Priority Data
Sep. 7, 2021 (KR) 10-2021-0118911

(51) Int. Cl. C07K 16/28; A61K 39/00; A61P 7/02

(52) U.S. Cl. CPC C07K 16/2866; A61P 7/02; A61K 2039/505; C07K 2317/76

(57) ABSTRACT

Disclosed is novel use of granulocyte colony-stimulating factor receptor or colony-stimulating factor 3 receptor (CSF3R) as a biomarker and therapeutic target for pulmonary fibrosis. Herein, a treatment mechanism, treatment method, treatment drug, and therapeutic use of the treatment drug containing CSF3R for pulmonary fibrosis are disclosed.

### CROSS REFERENCE TO RELATED APPLICATION

The present application claims priority to Korean Patent Application No. 10-2021-0118911, filed Sep. 7, 2021, the entire contents of which is incorporated herein for all purposes by this reference.

### BACKGROUND OF THE DISCLOSURE

### 1. Field of the Disclosure

The present disclosure relates to a new use of granulocyte colony-stimulating factor receptor or colony-stimulating factor 3 receptor (CSF3R) as a biomarker and therapeutic target for pulmonary fibrosis. More specifically, the present disclosure relates to a kit for diagnosing pulmonary fibrosis and a method for diagnosing pulmonary fibrosis, wherein the kit and method involve measuring the expression level of CSF3R in a sample derived from lung tissue. The present disclosure also relates to a pharmaceutical composition for treating pulmonary fibrosis, the pharmaceutical composition containing a substance that inhibits the expression or activity of CSF3R.

### 2. Description of the Related Art

Pulmonary fibrosis is a disease in which chronic inflammatory cells infiltrate the alveoli, causing structural changes and fibrosis in lung tissue. When chronic inflammatory response occurs due to continuous stimulation or extreme damage to the lung tissue, the original function of the lung epithelial cells is lost. As extracellular matrix (ECM) including collagen and fibronectin accumulates around the damaged area, the tissue experiences fibrosis which causes the hardening of the tissue. When the lung tissue becomes hardened, gas exchange cannot occur smoothly, causing symptoms such as dyspnea. These symptoms have a significant impact on the patient's quality of life and prognosis.

The exact cause of pulmonary fibrosis has not yet been identified, but risk factors such as smoking, metal dust, wood dust, and solvents have been reported.

Due to the continuous increase in the prevalence of pulmonary fibrosis, the treatment market size is recording a high growth rate every year. However, existing treatments only delay the worsening of symptoms and cannot cure pulmonary fibrosis. Recently, Rangarajan, S. et al. reported that metformin, which is an oral diabetes treatment, can alleviate pulmonary fibrosis symptoms in a bleomycin (BLM)-induced pulmonary fibrosis mouse model (Rangarajan, S., et al. Metformin reverses established lung fibrosis in a bleomycin model. Nat Med. 2018. 24, 1121-1127). However, no significant results were obtained in actual clinical trials.

In addition, research results reported high side effects and low treatment effectiveness when anti-TGF-β therapy was administered for pulmonary fibrosis (Varga, J. & Pasche, B. Antitransforming growth factor-beta therapy in fibrosis: recent progress and implications for systemic sclerosis. Current opinion in rheumatology. 2008. 20, 720-728). Various studies have been reported targeting TGF-β which is a factor commonly expressed in fibrotic diseases of various organs. The target material itself not only regulates various signals within the human body but also causes side effects such as gastrointestinal symptoms and photosensitivity. Accordingly, there are difficulties with drug development.

The two drugs currently used for patients with pulmonary fibrosis are nintedanib (Ofev) and pirfenidone (Esbriet). However, the drugs only slow the progression of symptoms, have no curing effect, and cause many side effects. In particular, the average survival time of patients with pulmonary fibrosis is reported to be less than 3 years, and there is no effective treatment method other than lung transplantation.

Therefore, it is urgent to discover treatment targets for pulmonary fibrosis and develop therapeutic drugs based on the treatment targets.

### SUMMARY OF THE DISCLOSURE

### 1. Technical Problems to Solve

In this specification, the technical task is to discover a new therapeutic target for pulmonary fibrosis.

In this specification, the technical task is to provide a therapeutic drug for pulmonary fibrosis.

In this specification, the technical task is to provide a treatment method and mechanism for pulmonary fibrosis.

In this specification, the technical task is to provide the use of a treatment drug for pulmonary fibrosis.

According to the pulmonary fibrosis treatment drug, treatment method, and mechanism of the present disclosure, patients with pulmonary fibrosis can be effectively treated.

### 2. Modes of the Invention

In this specification, a method for treating pulmonary fibrosis comprising followings is provided:
administering a therapeutically effective amount of anti-CSF3R antibody to a patient in need of treatment,
wherein the anti-CSF3R antibody promotes a degradation of an accumulated extracellular matrix in the lung tissue of the patient.

In one embodiment, the promoting the degradation of the accumulated extracellular matrix comprises reducing (decreasing) an expression level of one or more markers selected from Col1a1, OPN, VER, FN, and Has3.

In one embodiment, the accumulated extracellular matrix is an accumulated collagen.

In one embodiment, the degradation of the accumulated collagen is promoted by at least one of the followings:
an increase (or increasing) of an expression of matrix metalloproteinase (MMP) in the lung tissue of the patient; and
a decrease (or decreasing) of an expression of tissue inhibitors of metalloproteinase (TIMP) in the lung tissue of the patient.

In one embodiment, the matrix metalloproteinase (MMP) is one or more selected from MMP2, MMP9, and MMP13.

In one embodiment, tissue inhibitors of the metalloproteinase (TIMP) is one or more selected from TIMP-1 and TIMP-2.

In this specification, method for treating pulmonary fibrosis comprising followings is provided:
administering a therapeutically effective amount of an anti-CSF3R antibody to a patient in need of treatment,
wherein the anti-CSF3R antibody performs a function selected from the followings, in a lung tissue of the patient:
inhibiting a CSF3R-mediated epithelial to mesenchymal transition (EMT) in a cell within the lung tissue of the patient; and
reverting a cell in the lung tissue of the patient, in which the CSF3R-mediated epithelial to mesenchymal transition (EMT) is occurred, into an epithelial cell or fibroblast.

In one embodiment, the inhibiting of CSF3R-mediated EMT, or the reverting a cell which the CSF3R-mediated EMT is occurred induces one or more selected from the followings:
decreasing an expression of one or more markers selected from Fibronectin, Vimentin, N-cad, and ZEB1; and
increasing an expression of the E-cad.

In one embodiment, the anti-CSF3R antibody is capable of inhibiting or decreasing a binding of STAT3 with CSR3R in a cell of the lung tissue of the patient.

In one embodiment, the pulmonary fibrosis is selected from an idiopathic pulmonary fibrosis, a pulmonary inflammatory fibrosis disease, a chronic obstructive pulmonary disease, and a fibrosis disease occurring in asthma.

In this specification, an anti-CSF3R antibody for use in treating patient's pulmonary fibrosis, wherein the anti-CSF3R antibody is capable of promoting a degradation of an accumulated extracellular matrix in the lung tissue of the patient, is provided.

In one embodiment, the promoting the degradation of the accumulated extracellular matrix means a decreasing an expression of one or more markers selected from Col1a1, OPN, VER, FN, and Has3.

In one embodiment, the accumulated extracellular matrix is an accumulated collagen.

In one embodiment, the degradation of the accumulated collagen is promoted by at least one of the followings:
an increase of an expression of matrix metalloproteinase (MMP) in the lung tissue of the patient; and
a decrease of an expression of tissue inhibitors of metalloproteinase (TIMP) in the lung tissue of the patient.

In one embodiment, the matrix metalloproteinase (MMP) is one or more selected from MMP2, MMP9, and MMP13.

In one embodiment, the tissue inhibitors of metalloproteinase (TIMP) is one or more selected from TIMP-1 and TIMP-2.

In this specification, an anti-CSF3R antibody for use in treating patient's pulmonary fibrosis, is provided,
wherein the anti-CSF3R antibody is capable of performing one or more functions selected from the followings:
inhibiting a CSF3R-mediated epithelial to mesenchymal transition (EMT) of a cell in the lung tissue of the patient; and
reverting a cell in the lung tissue of the patient, in which the CSF3R-mediated epithelial to mesenchymal transition (EMT) is occurred, into an epithelial cell or fibroblast.

In one embodiment, the inhibiting of CSF3R-mediated EMT is one or more selected from the followings:
decreasing an expression of one or more markers selected from Fibronectin, Vimentin, N-cad, and ZEB1; and increasing an expression of the E-cad.

In one embodiment, the anti-CSF3R antibody is capable of inhibiting or decreasing a binding of STAT3 with CSR3R in a cell of the lung tissue of the patient.

In one embodiment, the pulmonary fibrosis is selected from an idiopathic pulmonary fibrosis, a pulmonary inflammatory fibrosis disease, a chronic obstructive pulmonary disease, and a fibrosis disease occurring in asthma. □

### BRIEF DESCRIPTION OF THE DRAWINGS

FIG. 1 shows the results of confirming CSF3R expression in respective lung tissue of a normal person, and lung tissue of an idiopathic pulmonary fibrosis (IPF) patient using immunohistochemical staining;

FIG. 2 shows the results of confirming CSF3R expression in lung tissue of a normal person, and in lung tissue of an IPF patient, respectively, using immunohistochemical staining, the results being expressed as an immunohistochemical staining score (IHC Score);

FIG. 3 shows the results of confirming CSF3R expression in lung tissue of a normal person, and lung tissue of an IPF patient using GEO dataset;

FIG. 4 shows the results of confirming CSF3R expression in a lung tissue of a bleomycin (BLM)-induced IPF mouse model using immunohistochemical staining;

FIG. 5 shows the results of confirming CSF3R expression in a lung tissue of a BLM-induced IPF mouse model using western blotting;

FIG. 6 shows the results of confirming CSF3R expression in a lung tissue of a BLM-induced IPF mouse model using RT-qPCR;

FIG. 7 shows the results of RT-qPCR confirming an increase in the expression of extracellular matrix (ECM) components and epithelial-to-mesenchymal transition (EMT) markers when CSF3R is overexpressed in the lung epithelial cell line Beas-2b;

FIG. 8 shows the results of western blotting confirming an increase in the expression of extracellular matrix (ECM) components and epithelial-to-mesenchymal transition (EMT) markers when CSF3R is overexpressed in the lung epithelial cell line Beas-2b;

FIG. 9 shows the results of RT-qPCR confirming that the BLM-induced expression of extracellular matrix (ECM) components and epithelial-to-mesenchymal transition (EMT) markers are suppressed when CSF3R expression is inhibited in the lung epithelial cell line Beas-2b;

FIG. 10 shows the results of western blotting confirming that the BLM-induced expression of extracellular matrix (ECM) components and epithelial-to-mesenchymal transition (EMT) markers are suppressed when CSF3R expression is inhibited in the lung epithelial cell line Beas-2b;

FIG. 11 shows an experimental plan for the injection of CSF3R-neutralizing antibodies into a BLM-induced pulmonary fibrosis mouse model;

FIG. 12 shows the results of immunohistochemical staining confirming the treatment effect of pulmonary fibrosis depending on CSF3R neutralizing antibody treatment;

FIG. 13 shows the results of a hydroxyproline assay confirming the collagen reduction effect due to CSF3R neutralizing antibody treatment;

FIG. 14 is a photograph showing that BLM-induced hair loss in mice is recovered by CSF3R-neutralizing antibodies;

FIG. 15 is a graph showing that BLM-induced weight loss in mice is recovered by CSF3R-neutralizing antibodies;

FIG. 16 shows the results of immunohistochemical staining confirming a decrease in epithelial-to-mesenchymal transition (EMT) marker proteins in mice having been subjected to administration of CSF3R neutralizing antibodies;

FIG. 17 shows the results of RT-qPCR confirming a decrease in epithelial-to-mesenchymal transition (EMT) marker proteins in mice having been subjected to the administration of CSF3R-neutralizing antibodies;

FIG. 18 shows the results of immunohistochemical staining showing that the expression of MMP2, MMP9, and MMP13, which was decreased by BLM treatment, increased again by administration of CSF3R neutralizing antibodies in a BLM-induced pulmonary fibrosis mouse model;

FIG. 19 is a graph showing that the expression levels of MMP2, MMP9, and MMP13, which were decreased by BLM treatment in a BLM-induced pulmonary fibrosis mouse model, increased again by administration of CSF3R neutralizing antibodies;

FIG. 20 shows the results of immunohistochemical staining confirming that the expression of TIMP-1 and TIMP-2, which was increased by BLM treatment in a BLM-induced pulmonary fibrosis mouse model, was reduced to the level of normal lung tissue by administration of CSF3R neutralizing antibodies;

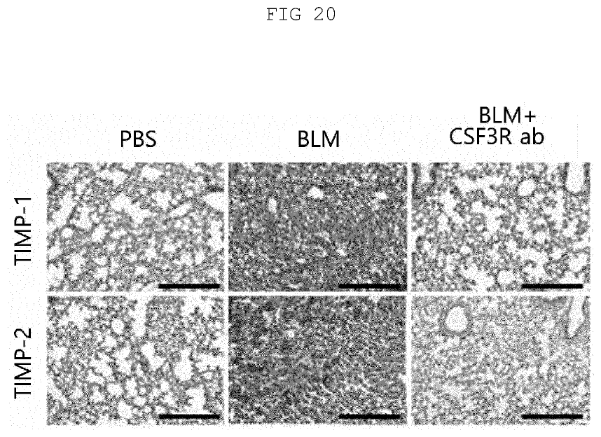

FIG. 21 shows the results of screening for downstream signaling pathway factors by administration of rh-CSF3 to lung epithelial cell line Beas-2b;

FIG. 22 shows the results of western blotting confirming that the BLM-induced activity of STAT3 decreases as the expression of CSF3R is inhibited;

FIG. 23 shows the results of co-immunoprecipitation (co-IP) confirming direct binding of CSF3R and STAT3;

FIG. 24 shows the results of an in-situ PLA assay confirming direct binding between CSF3R and STAT3; and

FIG. 25 shows the results of an in-situ PLA assay confirming direct binding between CSF3R and STAT3.

### DESCRIPTION OF THE PREFERRED EMBODIMENTS

Hereinafter, with reference to the attached drawings, the content of the disclosure will be described in more detail through specific implementation embodiments and examples. It should be noted that the attached drawings include some, but not all, embodiments of the disclosure. The subject matter of the disclosure disclosed by this specification may be implemented in various ways and is not limited to the specific implementation examples described herein. These implementations should be viewed as being provided to satisfy the legal requirements applicable to this specification. Those skilled in the art will be able to think of many modifications and other implementations of the disclosure disclosed herein. Accordingly, it should be understood that the content of the disclosure disclosed herein is not limited to the specific embodiments described herein and that modifications and other embodiments thereof are also included within the scope of the claims.

The present inventors confirmed that the expression of CSF3R was high in a patient with pulmonary fibrosis and a BLM-induced pulmonary fibrosis mouse model, and thus the present inventors suggest that CSF3R can be used as a biomarker for diagnosing pulmonary fibrosis. In addition, the present inventors confirmed that when CSF3R experiencing an increase in its expression in tissue of pulmonary fibrosis was inhibited by targeting antibodies, alpha-myofibrillar proteins, collagens, and epithelial-to-mesenchymal transition (EMT) marker factors were significantly reduced. Thus, the present inventors suggest that CSF3R may serve as an effective therapeutic target for pulmonary fibrosis.

Specifically, the present inventors observed that the expression of CSF3R was higher in tissue of pulmonary fibrosis than in normal lung tissue by using a human tissue microarray, GEO database, and BLM-induced pulmonary fibrosis mouse model.

In addition, an experiment was conducted to confirm changes in lung tissue by using H&E staining, and the experiment was conducted to compare the expression of markers for pulmonary fibrosis and epithelial-to-mesenchymal transition by using immunohistochemical staining. In addition, the present inventors verified that a treatment effect for pulmonary fibrosis appeared CSF3R antibodies were when targeting administered intraperitoneally into the BLM-induced pulmonary fibrosis mouse model.

In conclusion, CSF3R can be a biomarker for diagnosing pulmonary fibrosis and a new target for treating pulmonary fibrosis, and pulmonary fibrosis can be treated by inhibiting the expression or activity of CSF3R.

Based on the findings, in one aspect, the present disclosure relates to a kit for diagnosing pulmonary fibrosis including a reagent for measuring the expression level of CSF3R in a sample derived from lung tissue.

In another aspect, the present disclosure relates to a method of providing information for diagnosing pulmonary fibrosis, the method involving measuring the expression level of CSF3R in a sample derived from lung tissue.

In the kit for diagnosing pulmonary fibrosis and the method of providing information for diagnosing pulmonary fibrosis, when the expression level of CSF3R in the sample derived from lung tissue is higher than that in the sample derived from normal lung tissue of a control group, the sample derived from lung tissue can be determined to develop pulmonary fibrosis.

In the present disclosure, “pulmonary fibrosis” is a respiratory disease that causes the hardening of lung tissue and then serious breathing problems. The hardening of lungs means excessive accumulation of fibrous connective tissue, and this process is called fibrosis. As fibrosis progresses, the lung walls thicken and the amount of oxygen supplied to the blood decreases, resulting in patients continuously feeling short of breath. It is reported that there is no way to restore lung tissue that has developed fibrosis. In one embodiment, pulmonary fibrosis may be an idiopathic pulmonary fibrosis, a pulmonary inflammatory fibrosis disease, a chronic obstructive pulmonary disease, and a fibrosis disease occurring in asthma. Specifically, pulmonary fibrosis may be idiopathic pulmonary fibrosis.

The term “diagnosis” used in the present disclosure, in a broad sense, means judging the actual disease conditions of a patient in all aspects. The contents of the judgment include the name of the disease, etiology, type, severity, detailed conditions of the disease, and the presence or absence of complications. In the present disclosure, diagnosis refers to judging the onset and progression level of pulmonary fibrosis.

As used herein, the term “method of providing information for diagnosis of pulmonary fibrosis” refers providing objective and foundational information necessary for the diagnosis of pulmonary fibrosis, as a preliminary step for diagnosis or prognosis prediction, and excludes the clinical judgment or opinion of a doctor.

In the present disclosure, measuring the expression level of CSF3R in a sample derived from lung tissue involves measuring the mRNA expression level of the CSF3R gene or measuring the expression level of the CSF3R protein encoded by the gene.

Methods for measuring the mRNA expression level include, but are not limited to, reverse transcription polymerase reaction (RT-PCR), competitive reverse transcription polymerase reaction (Competitive RT-PCR), real-time reverse transcription polymerase reaction (Real-time RT-PCR), and RNase protection assay (RPA), northern blotting, and DNA chip.

Methods for measuring the expression level of the protein include, but are not limited to, western blotting, enzyme-linked immunosorbent assay (ELISA), radioimmunoassay (RIA), radioimmunodiffusion, ouchterlony, immunodiffusion, rocket immunoelectrophoresis, tissue immunostaining, immunoprecipitation assay, complement fixation assay, fluorescence activated cell sorter (FACS), and protein chip.

In yet another aspect, the present disclosure relates to a pharmaceutical composition for treating pulmonary fibrosis, the pharmaceutical composition containing a substance that inhibits the expression or activity of CSF3R. In one embodiment, the pharmaceutical composition of the present disclosure can inhibit the expression of α-Smooth Muscle Actin (α-SMA) in lung cells. In another embodiment, the pharmaceutical composition of the present disclosure can inhibit the expression of extracellular matrix including collagen in lung tissue. In yet another embodiment, the pharmaceutical composition of the present disclosure can inhibit the epithelial-to-mesenchymal transition (EMT) of lung epithelial cells. Specifically, the inhibition of epithelial-to-mesenchymal transition may include suppressing the expression of one or more proteins selected from the group consisting of fibronectin (FN), vimentin (VIM), N-cad, and ZEB1, increasing the expression of E-cad, or both.

As such, the pharmaceutical composition of the present disclosure inhibits the expression of alpha-Smooth Muscle Actin (α-SMA) in lung cells, and suppresses the expression of collagen in lung tissue; the composition does both and either one of inhibiting the epithelial-to-mesenchymal transition and restoring into lung epithelial cells from myofibroblast; or performs both. Thus, the pharmaceutical composition can effectively treat pulmonary fibrosis by ultimately reducing the accumulation of extracellular matrix components.

In the present disclosure, “epithelial-to-mesenchymal transition” refers to a process in which epithelial cells lose cell polarity and intercellular adhesion and gain migratory and invasive properties to become mesenchymal stem cells. The mesenchymal stem cells are multipotent stromal cells that can differentiate into various cell types. The epithelial-to-mesenchymal transition is essential for numerous developmental processes, including mesoderm formation and neural tube formation, and is observed during wound healing, organ fibrosis, and cancer metastasis. Therefore, pulmonary fibrosis can be treated by inhibiting and/or restoring the epithelial-to-mesenchymal transition of lung epithelial cells.

In the pharmaceutical composition for treating pulmonary fibrosis according to the present disclosure, a substance that inhibits the expression or activity of CSF3R may include CSF3R siRNA, CSF3R shRNA, CSF3R antisense nucleic acid, guide RNA (gRNA) and CRISPR/Cas9, anti-CSF3R small molecule compound, or anti-CSF3R antibodies.

Specifically, the substance that inhibits the expression of CSF3R includes CSF3R siRNA, CSF3R shRNA, CSF3R antisense nucleic acid, guide RNA (gRNA), and CRISPR/Cas9, which can specifically bind to the mRNA of CSF3R. Additionally, the substance that inhibits the activity of CSF3R includes anti-CSF3R small molecule compounds or anti-CSF3R antibodies that specifically bind to CSF3R and inhibit the activity of CSF3R.

In one embodiment, as a genetic method of inhibiting the expression of CSF3R in tissues of pulmonary fibrosis, the expression of CSF3R can be suppressed by using various carriers for siRNA, shRNA, or antisense nucleic acid all targeting the CSF3R gene or directly introducing the siRNA, shRNA, or antisense nucleic acid into pulmonary fibrosis lung tissue. Alternatively, guide RNA (gRNA) targeting the CSF3R gene and CRISPR/Cas9 can be introduced into pulmonary fibrosis lung tissue to delete or suppress the CSF3R gene. Additionally, a CRISPR/dCas9-based transcriptional repressor targeting the CSF3R gene can be introduced into pulmonary fibrosis lung tissue to inhibit the expression of the CSF3R gene.

In one embodiment, as a pharmacological method of inhibiting the expression of CSF3R in a pulmonary fibrosis cell, the expression of the CSF3R gene can be inhibited by injecting a small molecule compound, which inhibits transcription of CSF3R, into pulmonary fibrosis lung tissue.

In one embodiment, as a pharmacological method of inhibiting the activity of CSF3R in a pulmonary fibrosis cell, the activity of the CSF3R gene can be inhibited by injecting a small molecule compound, which inhibits the activity of CSF3R, into pulmonary fibrosis lung tissue.

siRNA (small interfering RNA) refers to short double-stranded RNA that can induce RNAi (RNA interference) phenomenon through cleavage of specific mRNA. siRNA includes a sense RNA strand having a sequence homologous to the mRNA of the target gene and an antisense RNA strand having a complementary sequence. Since siRNA can inhibit the expression of target genes, siRNA can be provided as an efficient gene knockdown method or as a method of gene therapy.

shRNA (short hairpin RNA or small hairpin RNA) is used to overcome the disadvantages of siRNA, such as the high biosynthesis cost and short-term maintenance of RNA interference effect due to low cell transfection efficiency. shRNA can be introduced into cells and expressed from the promoter of RNA polymerase III using adenovirus, lentivirus, and plasmid expression vector systems. Such shRNA is widely known to be converted into siRNA with an accurate structure by siRNA processing enzymes (Dicer or RNase III) present in a cell, thereby inducing silencing of the target gene.

Antisense nucleic acid refers to DNA or RNA, or derivatives thereof containing a nucleic acid sequence complementary to the sequence of a specific mRNA. The antisense nucleic acid can bind to complementary sequences within an mRNA and inhibit translation of the mRNA into protein, translocation into the cytoplasm, maturation, or any other essential activity for overall biological functions.

Antibodies that can specifically bind to CSF3R include monoclonal antibodies, chimeric antibodies, humanized antibodies, and human antibodies. The antibodies may also include antibodies already known in the art in addition to novel antibodies. The antibodies include functional fragments of the antibody molecules, as well as the full-length form of the two heavy and two light chains, insofar as the antibodies specifically bind to CSF3R. The functional fragments of the antibody molecules refer to fragments that possess at least an antigen-binding function, and include Fab, F(ab′), F(ab′)2, and Fv.

Methods of injecting a substance, which inhibits the expression or activity of CSF3R, into a pulmonary fibrosis cell can be implemented in various forms, such as using liposomes or vector systems which are a genetic engineering technology. Alternatively, a method of preparing a drug in the form of preparation and administering the drug into the body in a conventional manner may be used.

The pharmaceutical composition according to the present disclosure may further include a pharmaceutically acceptable carrier. The pharmaceutically acceptable carrier is commonly used in preparation and includes, but is not limited to, saline solution, sterile water, Ringer's solution, buffered saline solution, cyclodextrin, dextrose solution, maltodextrin solution, glycerol, ethanol, and liposome. The pharmaceutically acceptable carrier may further include other common additives such as an antioxidant and buffer, if necessary. The pharmaceutically acceptable carrier can also be formulated into an injectable formulation (for example, an aqueous solution, suspension, and emulsion), pill, capsule, granule, or tablet by additionally adding a diluent, dispersant, surfactant, binder, and lubricant. A suitable pharmaceutically acceptable carrier and preparation can be preferably formulated depending on each ingredient using the method disclosed in Remington's Pharmaceutical Sciences (19th edition, 1995). The pharmaceutical composition of the present disclosure is not particularly limited in formulation but can be formulated as an injection, inhalation agent, topical skin agent, or oral ingestion agent.

The composition of the present disclosure can be administered orally or parenterally (for example, intravenous area, subcutaneous area, skin, nasal cavity, or respiratory tract) depending on the desired method. Dosage varies depending on the patient's condition and weight, degree of disease, drug form, administration route, and time, but can be appropriately selected by a person skilled in the art.

The composition according to the present disclosure is administered in a therapeutically effective amount. In the present disclosure, “therapeutically effective amount” means an amount sufficient to treat a disease with a reasonable benefit/risk ratio applicable to medical treatment. The effective dose level can be decided depending on factors including the type and severity of the patient's disease, drug activity, sensitivity to the drug, time of administration, route of administration and excretion rate, duration of treatment, concurrently used drugs, and other factors well known in the field of medicine. The composition according to the present disclosure can be administered as an individual treatment or in combination with other treatments. In addition, the composition can be administered sequentially or simultaneously with conventional therapeutic agents and can be administered singly or multiple times. Considering all of the factors, it is important to administer the minimal amount that can achieve maximum effect with the fewest side effects, and the amount can be easily determined by a person skilled in the art.

Specifically, the effective amount of the composition according to the present disclosure may vary depending on the patient's age, gender, and weight. In general, 0.001 to 150 mg, preferably 0.01 to 100 mg, of the composition per kg of body weight can be administered every day or every other day, or the administration can be divided into 1 to 3 times per day. However, dosage does not limit the scope of the present disclosure in any way since the effective amount may increase or decrease depending on the route of administration, severity of pulmonary fibrosis, gender, weight, and age.

Meanwhile, the present inventors analyzed the expression of matrix metalloproteinases (MMPs) and tissue inhibitors of metalloproteinases (TIMPs) related to the degradation of extracellular matrix (ECM). The analysis was conducted to identify the mechanism by which BLM-induced PF-affected lung tissue is restored to normal lung tissue by CSF3R-neutralizing antibodies. As a result, as confirmed in the examples described antibodies were effective in later, the CSF3R-neutralizing treating pulmonary fibrosis in a BLM-induced pulmonary fibrosis mouse model by regulating the expression of MMPs (MMP2, MMP9, and MMP13) and TIMPs (TIMP-1 and TIMP-2). In addition, the present inventors further identified that STAT3 was a major factor in the downstream signaling mechanism that induced epithelial-to-mesenchymal transition (EMT) of lung epithelial cell lines by CSF3R.

In yet another aspect, the present disclosure relates to a method of screening a drug for treating pulmonary fibrosis including:
(1) treating cells with candidate substances, the cells being derived from a subject with pulmonary fibrosis;
(2) measuring the expression level of CSF3R in the cells; and
(3) selecting a substance as a therapeutic drug that reduces the expression level of CSF3R compared to the group not treated with the candidate substance.

In yet another aspect, the present disclosure provides a method of preventing or treating pulmonary fibrosis, the method including administering the composition to a subject.

The term “prevention” used herein refers to all actions that suppress or delay the onset of pulmonary fibrosis by administering the composition according to the present disclosure.

The term “treatment” used herein refers to any action in which symptoms of pulmonary fibrosis are improved or beneficially changed by administration of the composition according to the present disclosure.

In the present disclosure, “subject” means a subject in need of a method of preventing or treating a disease, more specifically, the term refers to mammals such as human or non-human primates, mice, rats, dogs, cats, horses, and cattle.

Additionally, the present disclosure presents CSF3R as a biomarker and novel therapeutic target for diagnosing pulmonary fibrosis.

Specifically, the present disclosure provides a kit for diagnosing pulmonary fibrosis including a reagent for measuring the expression level of CSF3R in a sample derived from lung tissue.

In addition, the present disclosure provides a method of providing information for diagnosing pulmonary fibrosis, the method including measuring the expression level of CSF3R in a sample derived from lung tissue.

The present disclosure also provides a pharmaceutical composition for treating pulmonary fibrosis, the pharmaceutical composition containing a substance that inhibits the expression or activity of CSF3R.

The present disclosure also provides a method of screening a drug for treating pulmonary fibrosis including:
(1) treating cells with candidate substances, the cells being derived from a subject with pulmonary fibrosis;
(2) measuring the expression level of CSF3R in the cells; and
(3) selecting a substance as a therapeutic drug that reduces the expression level of CSF3R compared to the group not treated with the candidate substance.

### Possible Examples of Disclosure

Possible examples of the disclosure provided in this specification are listed below. The following examples provided in this paragraph are merely examples of the disclosure. Therefore, the disclosure provided in this specification cannot be interpreted as limited to the following examples. The brief description provided along with the example number is only for the convenience of distinguishing between each example and cannot be construed as a limitation on the disclosure disclosed in this specification.

### Pulmonary Fibrosis

### Example 1. Pulmonary Fibrosis

Pulmonary fibrosis.

### Example 2. Subdivision of Pulmonary Fibrosis

In Example 1, pulmonary fibrosis is selected from an idiopathic pulmonary fibrosis, a pulmonary inflammatory fibrosis disease, a chronic obstructive pulmonary disease, and a fibrosis disease occurring in asthma.

### Example 3. Cause of Pulmonary Fibrosis

In any of Examples 1 to 2, the pulmonary fibrosis is bleomycin-induced pulmonary fibrosis or radiation-induced pulmonary fibrosis.

### Example 4. CSF3R Expression

In any one of Examples 1 to 3, in pulmonary fibrosis, the expression level of CSF3R in lung tissue of a patient with pulmonary fibrosis is increased compared to normal lung tissue.

### Example 5. Expression of Markers Related to Extracellular Matrix Degradation

In any one of Examples 1 to 4, in pulmonary fibrosis, the expression of markers selected from Col1a1, OPN, VER, FN, and Has3 in lung tissue of a patient with pulmonary fibrosis is increased compared to normal lung tissue.

### Example 6. Expression of Markers Related to Epithelial-to-Mesenchymal Transition

In any one of Examples 1 to 5, in pulmonary fibrosis, the expression of one or more markers selected from N-cad, FN, VIM, ZEB1, Snail, and Slug in lung tissue of a patient with pulmonary fibrosis is increased and/or the expression of an E-cad in lung tissue of a patient with pulmonary fibrosis is decreased compared to normal lung tissue.

### Therapeutic Agent 1-Anti-CSF3R Antibody

### Example 7. Anti-CSF3R Antibody

Anti-CSF3R antibodies.

### Example 8. Neutralizing Antibody

In Example 7, the anti-CSF3R antibodies are neutralizing antibodies against CSF3R.

### Example 9. Disruption of CSF3-CSF3R Binding

In any one of Examples 7 to 8, the anti-CSF3R antibodies block, interrupt, inhibit, and/or prevent the binding of CSF3 and CSF3R.

### Example 10. CSF3R Sequence Specification

In any one of Examples 7 to 9, the anti-CSF3R antibodies bind to the CSF3R protein represented by the amino acid sequence of

(SEQ ID NO: 1)
MARLGNCSLTWAALIILLLPGSLEECGHISVSAPIVHLGDPITASCIIKQ
NCSHLDPEPQILWRLGAELQPGGRQQRLSDGTQESIITLPHLNHTQAFLS
CCLNWGNSLQILDQVELRAGYPPAIPHNLSCLMNLTTSSLICQWEPGPET
HLPTSFTLKSFKSRGNCQTQGDSILDCVPKDGQSHCCIPRKHLLLYQNMG
IWVQAENALGTSMSPQLCLDPMDVVKLEPPMLRTMDPSPEAAPPQAGCLQ
LCWEPWQPGLHINQKCELRHKPQRGEASWALVGPLPLEALQYELCGLLPA
TAYTLQIRCIRWPLPGHWSDWSPSLELRTTERAPTVRLDTWWRQRQLDPR
TVQLFWKPVPLEEDSGRIQGYVVSWRPSGQAGAILPLCNTTELSCTFHLP
SEAQEVALVAYNSAGTSRPTPVVFSESRGPALTRLHAMARDPHSLWVGWE
PPNPWPQGYVIEWGLGPPSASNSNKTWRMEQNGRATGELLKENIRPFQLY
EIIVTPLYQDTMGPSQHVYAYSQEMAPSHAPELHLKHIGKTWAQLEWVPE
PPELGKSPLTHYTIFWTNAQNQSFSAILNASSRGFVLHGLEPASLYHIHL
MAASQAGAINSTVLILMILTPEGSELHIILGLFGLLLLLTCLCGTAWLCC
SPNRKNPLWPSVPDPAHSSLGSWVPTIMEEDAFQLPGLGTPPITKLTVLE
EDEKKPVPWESHNSSETCGLPTLVQTYVLQGDPRAVSTQPQSQSGTSDQV
LYGQLLGSPTSPGPGHYLRCDSTQPLLAGLTPSPKSYENLWFQASPLGTL
VTPAPSQEDDCVFGPLLNFPLLQGIRVHGMEALGSF.

### Example 11. Disruption of CSF3R-CSF3 Junction Specification

In Example 10, the anti-CSF3R antibodies bind to all or part of the CSF3R proteins selected from the following regions:
a region containing 190th arginine to 196th tyrosine in the amino acid sequence represented by SEQ ID NO: 1; and
a region containing 161th histidine to 314th leucine in the amino acid sequence represented by SEQ ID NO: 1.

### Example 12. Specification of Binding Target CSF3R-CSF3 Amino Acid

In Example 11, the anti-CSF3R antibodies bind to one or more amino acids in the amino acid sequence represented by SEQ ID NO: 1, the amino acids being selected from 190^{th} arginine, 194^{th} leucine, 195^{th} leucine, 196^{th} tyrosine, 261^{st} histidine, 262^{nd} isoleucine, 263^{rd} asparagine, and 314^{th} leucine.

### Therapeutic Agent 2-CSF3R Inhibitor

### Example 13. CSF3R Inhibitory Nucleic Acids

CSF3R inhibitors selected from CSF3R siRNA, CSF3R shRNA, and CSF3R antisense nucleic acid.

### Example 14. Binding Specifically to CSF3R mRNA

In Example 13, the CSF3R inhibitors are nucleic acids that specifically bind to the mRNA of the CSF3R gene.

### Example 15. CRISPR/Cas Inhibiting CSF3R

CRISPR/Cas composition targets the CSF3R gene, the composition including:
Cas protein or nucleic acid encoding the Cas protein; and
a guide RNA targeting the CSF3R gene or a nucleic acid encoding the guide RNA.

### Example 16. Limit to Cas Type

In Example 15, the Cas protein is Cas9 protein or Cas12a (Cpf1) protein.

### Example 17. Limit to Cas Function

In any one of Examples 15 to 16, the Cas protein has nickase activity or no nucleic acid cleavage activity (dead-form).

### Pharmaceutical Composition

### Example 18. Pharmaceutical Composition

A pharmaceutical composition for treating pulmonary fibrosis in any of Examples 1 to 6, the pharmaceutical composition including:
a therapeutically effective amount anti-CSF3R antibodies of any of Examples 7 to 12 and/or the CSF3R inhibitors of any of Examples 13 to 17; and
a pharmaceutically acceptable carrier and/or adjuvant.

### Example 19. Limit to Carrier Type

In Example 18, the pharmaceutically acceptable carrier is one or more selected from the following:
saline solution; sterile water; Ringer's solution; buffered saline; cyclodextrin; dextrose solution; maltodextrin solution; glycerol; ethanol; liposome; and additives such as antioxidants and buffers.

### Treatment Mechanism 1-Degradation Mechanism of Extracellular Matrix

### Example 20. Degradation Mechanism of Extracellular Matrix

A degradation mechanism of extracellular matrix.

### Example 21. Degradation Mechanism of Extracellular Matrix Related to CSF3R Inhibition

In Example 20, the degradation mechanism of the extracellular matrix is a CSF3R-mediated extracellular matrix degradation mechanism.

### Example 22. Collagen Degradation

In any one of Examples 20 to 21, the extracellular matrix subjected to the degradation mechanism of the extracellular matrix includes collagen.

### Example 23. Accumulated Collagen Degradation

In Example 22, the collagen is collagen accumulated in tissues.

### Example 24. Markers Related to Extracellular Matrix Degradation

In any one of Examples 20 to 23, the extracellular matrix degradation mechanism is that the expression of markers selected from Col1a1, OPN, VER, FN, and Has3 is blocked, reduced/decreased, inhibited, interrupted, and/or prevented.

### Example 25. MMPs, TIMPs

In any one of Examples 20 to 24, the degradation mechanism of the extracellular matrix involves promoting, activating, triggering, inducing, and/or causing one or more of the following:
an increase in expression level of matrix metalloproteinases (MMPs); and
a decrease in expression level of tissue inhibitors of metalloproteinases (TIMPs).

### Example 26. Additional Limit to Type of MMPs

In Example 25, the matrix metalloproteinases are one or more selected from MMP2, MMP9, and MMP13.

### Example 27. Additional Limit to TIMP Type

In any one of Examples 25 to 26, the tissue inhibitors of metalloproteinases are one or more selected from TIMP-1 and TIMP-2.

Treatment mechanism 2-Epithelial-to-mesenchymal transition mechanism

### Example 28. Inhibition of Epithelial-to-Mesenchymal Transition

A CSF3R-mediated epithelial-to-mesenchymal transition mechanism.

### Example 29. Markers Related to Epithelial-to-Mesenchymal Transition

In Example 28, the CSF3R-mediated epithelial-to-mesenchymal transition mechanism occurs by one or more selected from the following:
an increased expression of one or more markers selected from N-cad, FN, VIM, ZEB1, Snail, and Slug; and
a decreased expression of E-cad.

### Example 30. Submechanism 1-CSF3-CSF3R Binding

In any one of Examples 28 to 29, the CSF3R-mediated epithelial-to-mesenchymal transition mechanism is promoted, activated, triggered, induced, and/or caused by the binding of CSF3 and CSF3R.

### Example 31. Submechanism 2-CSF3R-STAT3 Binding

In any one of Examples 28 to 30, the CSF3R-mediated epithelial-to-mesenchymal transition mechanism is promoted, activated, triggered, induced, and/or caused by the binding of CSF3 and STAT3.

### Example 32. Submechanism 3-CSF3>CSF3R>CSF3R Binding

In any one of Examples 28 to 31, the CSF3R-mediated epithelial-to-mesenchymal transition mechanism is promoted, activated, triggered, induced, and/or caused by the following:
the binding and activation of CSF3 and CSF3R; and
the binding and activation of the activated CSF3R and STAT3.

### Pulmonary Fibrosis Treatment Methods

### Example 33. Pulmonary Fibrosis Treatment Method 1-Degradation of Extracellular Matrix

A treating method for pulmonary fibrosis of any one of Examples 1 to 6, comprising:
administering and injecting the anti-CSF3R antibodies of any one of Examples 7 to 12 or the CSF3R expression inhibitors of any of Examples 13 to 17 to a patient with pulmonary fibrosis, and/or delivering the anti-CSF3R antibodies of any one of Examples 7 to 12 or the CSF3R expression inhibitors of any of Examples 13 to 17 to the lung tissue of the patient.

Herein, the anti-CSF3R antibodies promote, activate, trigger, induce, and/or cause the extracellular matrix degradation mechanism of any one of Examples 20 to 27 within the lung tissue of the patient with pulmonary fibrosis.

### Example 34. Pulmonary Fibrosis Treatment Method 2 Inhibition/Restoration of Epithelial-to-Mesenchymal Transition

A treating method for pulmonary fibrosis of any one of Examples 1 to 6, comprising:
administering and injecting the anti-CSF3R antibodies of any one of Examples 7 to 12 or the CSF3R expression inhibitors of any of Examples 13 to 17 to a patient with pulmonary fibrosis, and/or delivering the anti-CSF3R antibodies of any one of Examples 7 to 12 or the CSF3R expression inhibitors of any of Examples 13 to 17 to the lung tissue of the patient.

Herein, the anti-CSF3R antibodies block, inhibit, interrupt, prevent, and/or reverse the CSF3R-mediated epithelial-to-mesenchymal transition mechanism of any one of Examples 28 to 32 within the lung tissue of the patient with pulmonary fibrosis.

### Example 35. Pulmonary Fibrosis Treatment Method 3-Combination of Treatment Mechanisms

A treating method for pulmonary fibrosis of any one of Examples 1 to 6, comprising:
administering and injecting the anti-CSF3R antibodies of any one of Examples 7 to 12 or the CSF3R expression inhibitors of any of Examples 13 to 17 to a patient with pulmonary fibrosis, and/or delivering the anti-CSF3R antibodies of any one of Examples 7 to 12 or the CSF3R expression inhibitors of any of Examples 13 to 17 to the lung tissue of the patient.

Herein, the anti-CSF3R antibodies block, inhibit, interrupt, prevent, and/or reverse the CSF3R-mediated epithelial-to-mesenchymal transition mechanism of any one of Examples 28 to 32 within the lung tissue of the patient with pulmonary fibrosis. The anti-CSF3R antibodies promote, activate, trigger, induce, and/or cause the collagen removal mechanism of any one of Examples to 27 within the lung tissue of the patient with pulmonary fibrosis.

### Example 36. Restoration of Epithelial-to-Mesenchymal Transition

In any one of Examples 34 to 35, the reversal of the epithelial-to-mesenchymal transition mechanism means the restoration of a cell into an epithelial cell or fibroblast, the cell experiencing the occurrence of epithelial-to-mesenchymal transition.

### Extracellular Matrix Degradation Method

### Example 37. Promotion Method for Extracellular Matrix Degradation

A method for promoting, activating, triggering, inducing, and/or causing the extracellular matrix degradation mechanism of any one of Examples 20 to 27 in lung tissue experiencing the occurrence of pulmonary fibrosis in any one of Examples 1 to 6 includes:
blocking, reducing/decreasing, inhibiting, interrupting, and/or preventing the function of CSF3R or the expression of CSF3R.

### Example 38. Method Limit 1-CSF3R Neutralization

In Example 37, the method is performed and/or induced by neutralizing CSF3R.

### Example 39. Antibody Use

In Example 38, the method includes introducing, delivering, injecting, and/or administering the anti-CSF3R antibodies of any one of Examples 7 to 12 into lung tissue experiencing the occurrence of pulmonary fibrosis.

### Example 40. Method Limit 2-Inhibition of CSF3R Expression

In Example 37, the method includes blocking, reducing/decreasing, inhibiting, interrupting, and/or preventing the expression of the CSF3R.

### Example 41. Use of CSF3R Inhibitors

In Example 40, the method includes introducing, delivering, injecting, and/or administering the CSF3R inhibitors of any one of Examples 13 to 17 into lung tissue experiencing the occurrence of pulmonary fibrosis.

### Epithelial-to-Mesenchymal Transition Inhibition/Recovery Method

### Example 42. Inhibition Method of Epithelial-to-Mesenchymal Transition

A method for blocking, inhibiting, interrupting, preventing, and/or reversing the epithelial-to-mesenchymal transition mechanism of any one of Examples 28 to 32 in lung tissue experiencing the occurrence of pulmonary fibrosis in any one of Examples 1 to 6 includes:
blocking, reducing/decreasing, inhibiting, interrupting, and/or preventing the function of CSF3R or the expression of CSF3R.

### Example 43. Restoration of Epithelial-to-Mesenchymal Transition

In Example 42, the restoration of the epithelial-to-mesenchymal transition mechanism means the restoration of a cell into an epithelial cell or fibroblast, the cell experiencing the occurrence of epithelial-to-mesenchymal transition.

### Example 44. Method Limit 1-CSF3R Neutralization

In Example 42, the method is performed and/or induced by neutralizing the CSF3R.

### Example 45. Antibody Use

In Example 44, the method includes introducing, delivering, injecting, and/or administering the anti-CSF3R antibodies of any one of Examples 7 to 12 into lung tissue experiencing the occurrence of pulmonary fibrosis.

### Example 46. Method Limit 2-Inhibition of CSF3R Expression

In Example 42, the method includes blocking, reducing/decreasing, inhibiting, interrupting, and/or preventing the expression of the CSF3R.

### Example 47. Use of CSF3R Inhibitor

In Example 46, the method includes introducing, delivering, injecting, and/or administering the CSF3R inhibitors of any one of Examples 13 to 17 into lung tissue experiencing the occurrence of pulmonary fibrosis.

### Use of Therapeutic Substances 1

### Example 48. Antibody Use

Use of the anti-CSF3R antibodies of any one of Examples 7 to 12 for treating pulmonary fibrosis in any of Examples 1 to 6.

The anti-CSF3R antibodies have one or more functions selected from the following:
the promotion, activation, trigger, induction, and/or cause of the extracellular matrix degradation mechanism of any one of Examples 20 to 27; and
the blocking, inhibition, interruption, prevention, and/or reversion of the CSF3R-mediated epithelial-to-mesenchymal transition mechanism of any one of Examples 28 to 32.

### Example 49. Restoration of Epithelial-to-Mesenchymal Transition

In Example 48, among the functions of the anti-CSF3R antibodies, the reversal of the epithelial-to-mesenchymal transition mechanism means the restoration of a cell into an epithelial cell or fibroblast, the cell experiencing the occurrence of epithelial-to-mesenchymal transition.

### Example 50. Use of CSF3R Inhibitors

Use of the CSF3R inhibitors of any of Examples 13 to 17 for treating pulmonary fibrosis of any of Examples 1 to 6.

The CSF3R inhibitors have one or more functions selected from the following:
the promotion, activation, trigger, induction, and/or cause of the extracellular matrix degradation mechanism of any one of Examples 20 to 27; and
the blocking, inhibition, interruption, prevention, and/or reversion of the CSF3R-mediated epithelial-to-mesenchymal transition mechanism of any one of Examples 28 to 32.

### Example 51. Restoration of Epithelial-to-Mesenchymal Transition

In Example 50, among the functions of the CSF3R inhibitors, the reversal of the epithelial-to-mesenchymal transition mechanism means the restoration of a cell into an epithelial cell or fibroblast, the cell experiencing the occurrence of epithelial-to-mesenchymal transition.

### Use of Therapeutic Substances 2

### Example 52. Antibody Use

Use of the anti-CSF3R antibodies in any one of Examples 7 to 12 for preparing a medicine for treating pulmonary fibrosis in any of Examples 1 to 6.

### Example 53. Limit to Treatment Mechanism

In Example 51, the anti-CSF3R antibodies have one or more functions selected from the following:
the promotion, activation, trigger, induction, and/or cause of the extracellular matrix degradation mechanism of any one of Examples 20 to 27; and
the blocking, inhibition, interruption, prevention, and/or reversion of the CSF3R-mediated epithelial-to-mesenchymal transition mechanism of any one of Examples 28 to 32.

### Example 54. Restoration of Epithelial-to-Mesenchymal Transition

In Example 53, among the functions of the anti-CSF3R antibodies, the reversal of the epithelial-to-mesenchymal transition mechanism means the restoration of a cell into an epithelial cell or fibroblast, the cell experiencing the occurrence of epithelial-to-mesenchymal transition.

### Example 55. Use of CSF3R Inhibitor

Use of the CSF3R inhibitors of any one of Examples 13 to 17 for preparing a medicine for treating pulmonary fibrosis in any of Examples 1 to 6.

### Example 56. Limit to Treatment Mechanism

In Example 55, the CSF3R inhibitors have one or more functions selected from the following:
the promotion, activation, trigger, induction, and/or cause of the extracellular matrix degradation mechanism of any one of Examples 20 to 27; and
the blocking, inhibition, interruption, prevention, and/or reversion of the epithelial-to-mesenchymal transition mechanism of any one of Examples 28 to 32.

### Example 57. Restoration of Epithelial-to-Mesenchymal Transition

In Example 56, among the functions of the CSF3R inhibitors, the reversal of the epithelial-to-mesenchymal transition mechanism means the restoration of a cell into an epithelial cell or fibroblast, the cell experiencing the occurrence of epithelial-to-mesenchymal transition.

### Experiment Examples

Hereinafter, the present disclosure provided by this specification will be described in more detail through experimental examples and examples. These examples are intended solely to illustrate the content disclosed by this specification. It will be apparent to those skilled in the art that the scope of the content disclosed by this specification is not to be construed as limited by these examples.

### Experiment Example 1. Experiment Preparation and Experiment Method

### Experiment Example 1.1. Cell Culture

Epithelial cell line Beas2B was cultured in RPMI Invitrogen media supplemented with 10% fetal bovine serum (FBS).

### Experiment Example 1.2. Transfection

Vectors and siRNA were transfected into the cells of cell line Beas2B using LipofectamineTM 2000 (Invitrogen) according to the provided manual.

### Experiment Example 1.3. Western Blotting

Proteins were isolated from the cells using lysis buffer (40 mM Tris-HCl (pH 8.0), 120 mM NaCl, 0.1% Nonidet-P40) which had been added with protease inhibitors. The proteins were separated by having been transferred to an SDS-PAGE and nitrocellulose membrane (Amersham, Arlington Heights, IL). The membrane was blocked with 5% nonfat dry milk (in Tris-buffered saline), and then the membrane reacted with primary antibodies at a temperature of 4° C. The membrane reacted with peroxidase-conjugated secondary antibodies and was visualized using enhanced chemiluminescence (ECL, Amersham, Arlington, Heights, IL).

### Experiment Example 1.4. Real-Time Quantitative PCR

RNA was extracted using trizol (Invitrogen, Carlsbad, CA, USA). qRT-PCR was performed using the SensiFAST TM SYBR NO-ROX Kit (Bioline Reagents, UK). Reactions were performed using otor Gene Q (Qiagene, Seoul, Korea). The results were calculated using the AACt method and normalized to β-actin.

### Experiment Example 1.5. Immunohistochemistry (IHC)

Mouse tissues were fixed with formalin and made into paraffin blocks. The paraffin-embedded tissue was cut and the paraffin was removed using xylene, 100%, 95%, 80%, and 70% ethanol, respectively. The cut tissue was subjected to hematoxylin and eosin (H&E) staining, Sirius red staining (abc150681), and DAB staining, respectively. In the DAB staining process, the tissue reacted with primary antibodies at a temperature of 4° C. overnight and then reacted with biotinylated secondary antibodies and ABC reagent (Vector Laboratories, USA) for 1 hour each. 3, 3-diaminobenzidine (Vector Laboratories) was used for the staining reaction, and counterstaining was performed with hematoxylin. Afterward, the tissue reacted with 70%, 80%, 95%, and 100% of ethanol, and xylene, respectively, and then was mounted on Canada balsam mounting media. Images were observed by using the DP71 system of an IX71 microscope (Olympus, Seoul, Korea).

### Experiment Example 1.6 Human Tissue Microarray

US-Biomax (LC561) was purchased as the microarray sample of human lung tissue of interstitial fibrosis, and the expression of CSF3R in the sample was analyzed through IHC.

### Experiment Example 1.7. Data Analysis

A gene expression omnibus (GEO) dataset (GSE71351) was used to analyze the genome of a patient with pulmonary fibrosis.

### Experiment Example 1.8. Animal Experiment

To create a model of pulmonary fibrosis, 100 mg/kg of bleomycin sulfate was intraperitoneally injected into male C57BL/6 mice four times (Days 1, 4, 7, and 10). After pulmonary fibrosis occurred, 250 μg/kg of neutralizing antibodies were injected intraperitoneally four times (Days 12, 14, 16, and 18), and on Day 21, the mouse was sacrificed and the lung tissue of the mouse was extracted.

### Experiment Example 1.9. Hydroxyproline Assay

Hydroxyproline levels were analyzed in mouse tissue lysates using a hydroxyproline assay kit (ab222941, abcam, UK). The experiment was conducted based on the manufacturer's instructions.

### Experiment Example 1.10. Co-Immunoprecipitation

Cell proteins extracted with lysis buffer reacted with primary antibodies overnight at a temperature of 4° C. Afterward, the cell proteins were precipitated using protein A-agarose beads (Santa Cruz Biotechnology, Inc.). The protein A-agarose beads were washed with cold PBS, and the precipitated proteins were analyzed through western blotting.

### Experiment Example 1.11. In-Situ Proximity Ligation Assay (PLA)

Cells cultured on coverslips were fixed with 4% paraformaldehyde and then permeabilized with a PBS solution containing 0.1% Triton X-100 and 10% fetal bovine serum. After the cells reacted with primary antibodies such as CSF3R antibody and STAT3 antibody overnight, a PLA experiment was performed with the cells using Duolink Detection Kit (Sigma). Visualization was performed through confocal microscopy.

### Experiment Example 2. Expression Analysis of CSF3R in Tissue of Patient with Idiopathic Pulmonary Fibrosis and Idiopathic Pulmonary Fibrosis Mouse Model

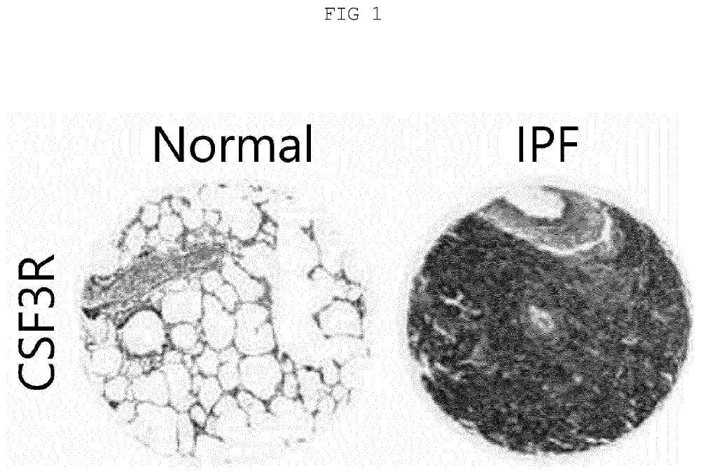

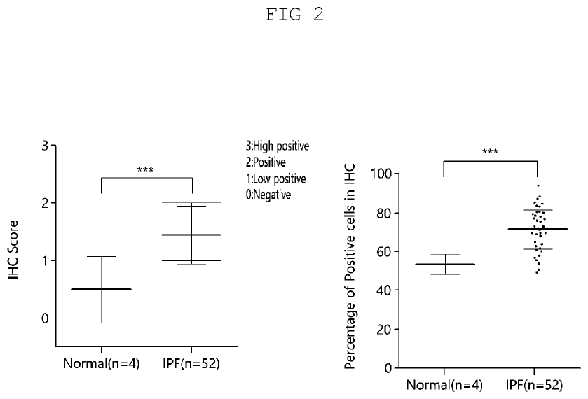

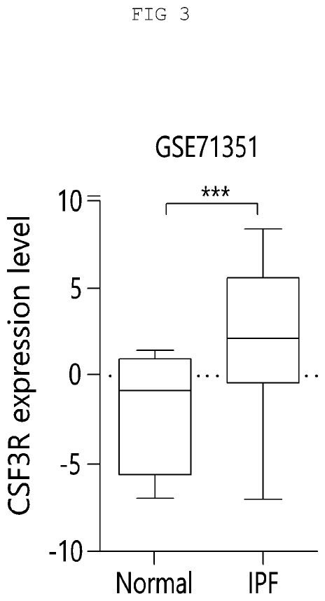

The expression of CSF3R was analyzed by using immunohistochemical staining (IHC) on tissue microarray results from a patient with idiopathic pulmonary fibrosis (IPF). As a result, it was observed that CSF3R was highly expressed in the tissue of patients with pulmonary fibrosis (see immunohistochemical staining results in FIG. 1 and IHC score results in FIG. 2). Additionally, using the GEO dataset, it was confirmed that the expression of CSF3R was increased in the tissue of a patient with idiopathic pulmonary fibrosis (IPF) compared to normal lung tissue (see FIG. 3).

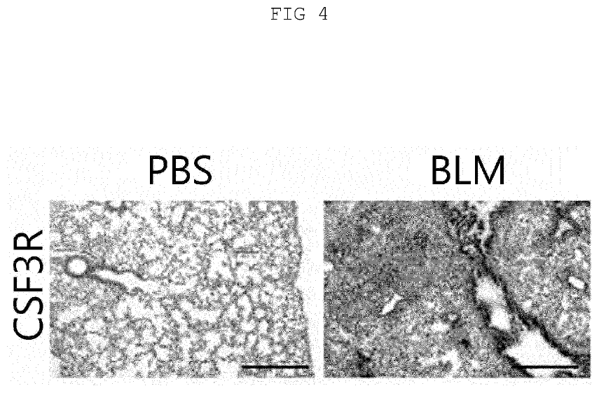

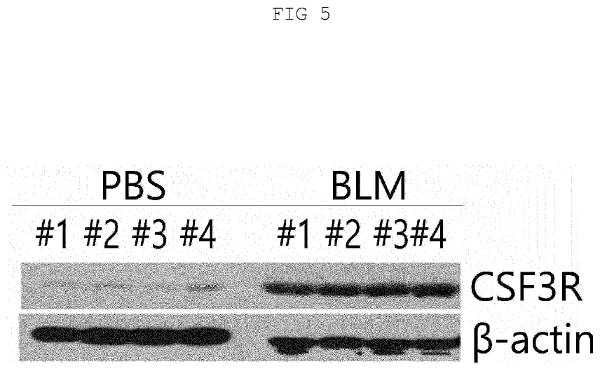

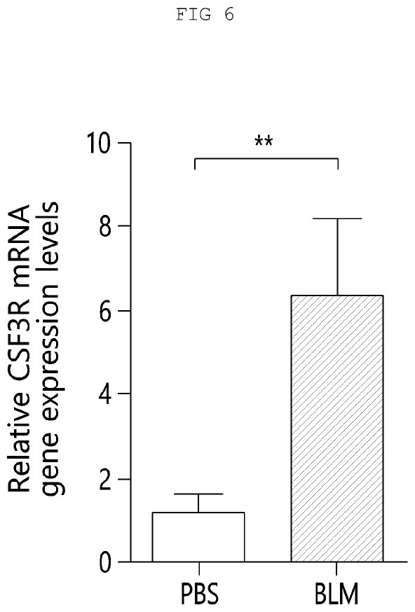

In addition, the expression level of CSF3R in lung tissue of a pulmonary fibrosis mouse model, especially a bleomycin (BLM)-induced idiopathic pulmonary fibrosis mouse model, was confirmed through immunohistochemical staining, western blotting, and RT-qPCR, respectively. The results are shown in FIGS. 4, 5, and 6, respectively. As shown in FIGS. 4 to 6, the expression of CSF3R increased in the BLM-induced IPF-affected lung tissue of the mouse model.

Through the experiment, it was confirmed that CSF3R was highly expressed in a patient with pulmonary fibrosis and a pulmonary fibrosis mouse model.

### Experiment Example 3. CSF3R-Induced EMT and Pulmonary Fibrosis in Lung Epithelial Cells

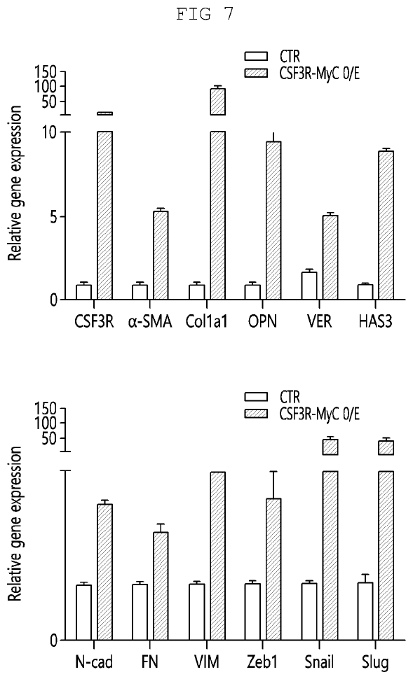

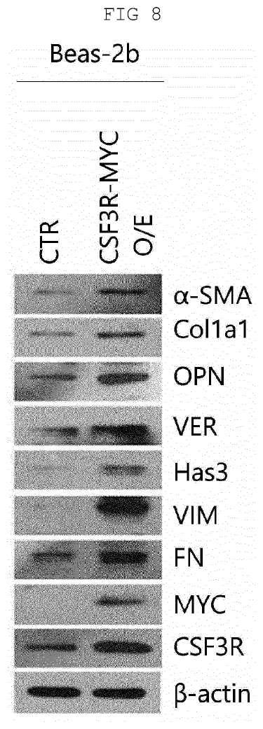

When CSF3R was overexpressed in lung epithelial cell line Beas-2b, the effects on extracellular matrix (ECM) components and epithelial-to-mesenchymal transition (EMT) markers were confirmed by RT-qPCR and western blotting. The results are shown in FIGS. 7 and 8, respectively. As shown in FIGS. 7 and 8, when CSF3R was overexpressed in lung epithelial cell line Beas-2b, the expression of extracellular matrix (ECM) components (x-SMA, Col1a1, OPN, VER, and HAS3) and epithelial-to-mesenchymal transition (EMT) markers (N-cad, FN, VIM, Zeb1, Snail, and Slug) increased.

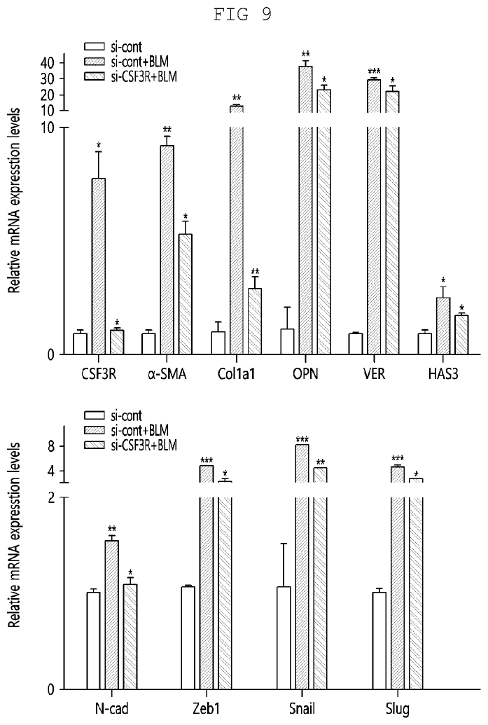

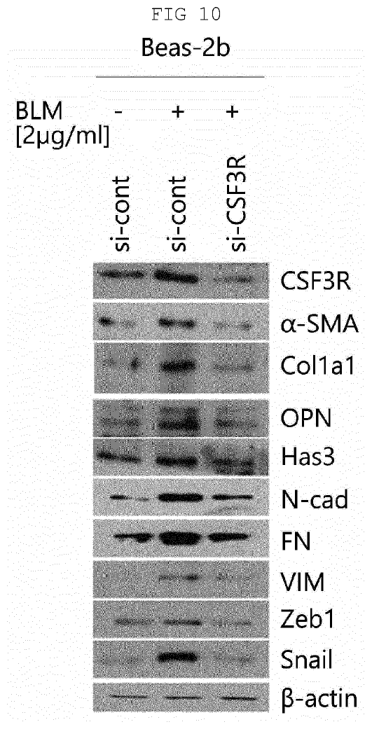

In addition, when inhibiting the expression of CSF3R in cell line Beas-2b, it was confirmed through RT-qPCR and western blotting that whether the BLM-induced expression of epithelial-to-mesenchymal transition (EMT) markers and extracellular matrix (ECM) components was inhibited. As shown in FIGS. 9 and 10, when the expression of CSF3R was inhibited in cell line Beas-2b, the BLM-induced expression of epithelial-to-mesenchymal transition (EMT) markers and extracellular matrix (ECM) components was inhibited.

Through the experiment, it was confirmed that CSF3R could induce pulmonary fibrosis by regulating the epithelial-to-mesenchymal transition (EMT) of lung epithelial cells.

### Experiment Example 4. Treatment Effect of Pulmonary Fibrosis Through CSF3R Neutralizing Antibody Treatment

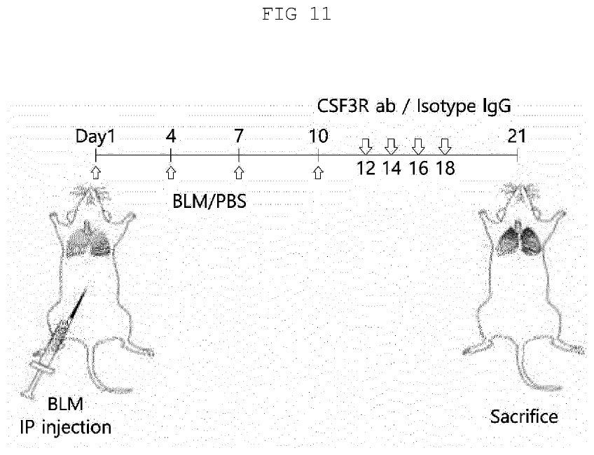

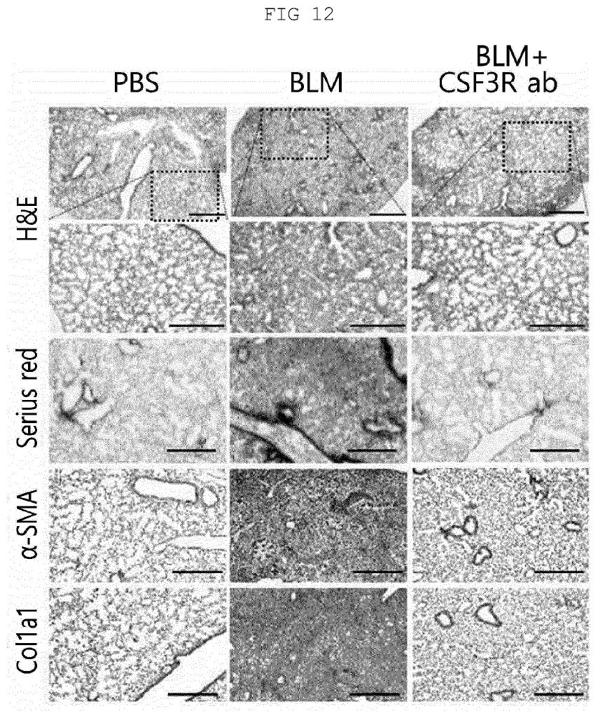

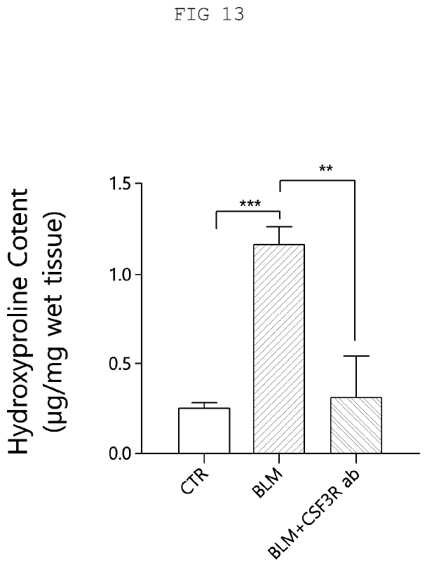

To confirm the effect of inhibiting CSF3R on treating pulmonary fibrosis, CSF3R-neutralizing antibodies were intraperitoneally injected four times into a BLM-induced pulmonary fibrosis mouse model. The specific plan for a CSF3R-neutralizing antibody injection experiment is shown in FIG. 11. It was observed through immunohistochemical staining that the symptoms of BLM-induced pulmonary fibrosis were alleviated in the group treated with CSF3R neutralizing antibodies (see FIG. 12). In addition, hydroxyproline assay was performed to determine whether the collagen seeing a BLM-induced increase thereof in lung tissue was decreased by CSF3R neutralizing antibody treatment. The results are shown in FIG. 13. As shown in FIG. 13, collagen seeing a BLM-induced increase thereof in lung tissue was significantly decreased by CSF3R neutralizing antibody treatment.

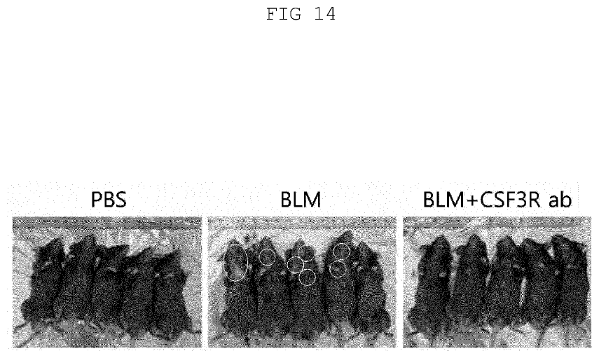

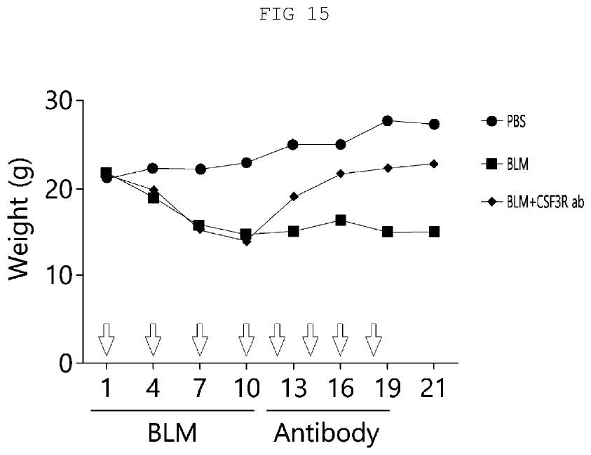

In addition, it was confirmed whether BLM-induced hair loss and weight loss in a mouse model could be recovered by CSF3R-neutralizing antibody treatment. As a result, as shown in FIG. 14, BLM-induced hair loss in a mouse model was recovered by CSF3R-neutralizing antibody treatment. As a result, as shown in FIG. 15, BLM-induced weight loss in a mouse model was recovered by CSF3R-neutralizing antibody treatment.

Through the experiment, it was confirmed that inhibiting CSF3R had a therapeutic effect on pulmonary fibrosis.

### Experiment Example 5. Verification of Inhibiting BLM-Induced EMT in Lung Tissue of Mouse Model Through CSF3R Neutralizing Antibody Treatment

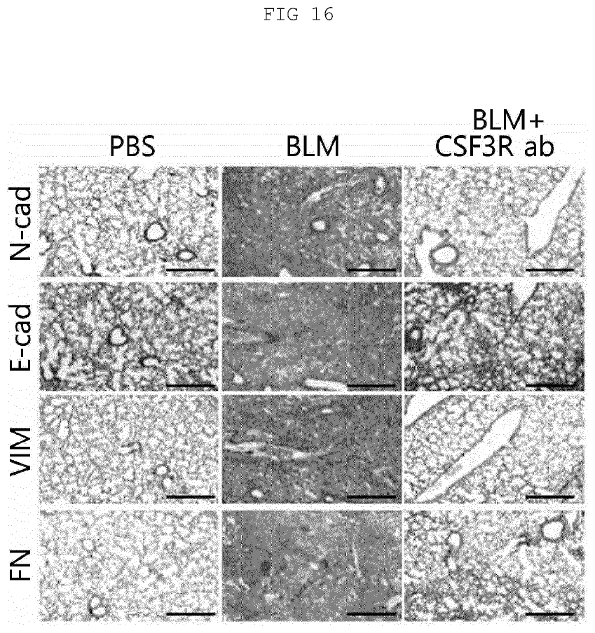

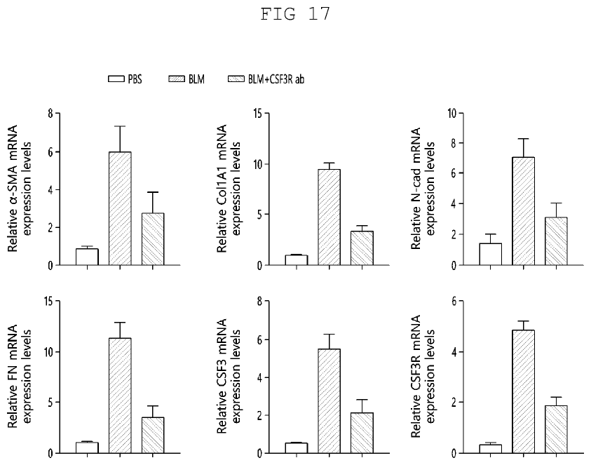

Previously, in Experiment Example 3, it was confirmed that the epithelial-to-mesenchymal transition (EMT) of lung epithelial cells was regulated by CSF3R. Accordingly, immunohistochemical staining and RT-qPCR were performed to confirm whether the epithelial-to-mesenchymal transition (EMT) was inhibited in pulmonary fibrosis lung tissue when CSF3R was inhibited in a mouse model. As a result, it was verified that the marker proteins of epithelial-to-mesenchymal transition (EMT) were decreased in the mouse model injected with CSF3R neutralizing antibodies (see immunohistochemical staining results in FIG. 16 and RT-qPCR results in FIG. 17).

Through the experiment, it was confirmed that EMT in pulmonary fibrosis lung tissue was inhibited when CSF3R was inhibited.

### Experiment Example 6. Identification of Pulmonary Fibrosis Treatment Mechanism by CSF3R Antibodies

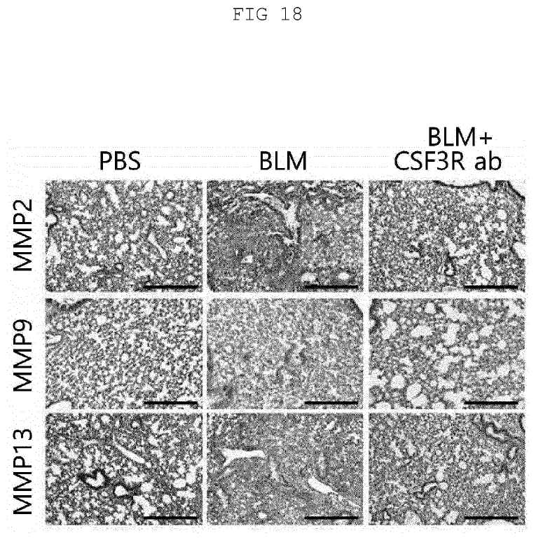

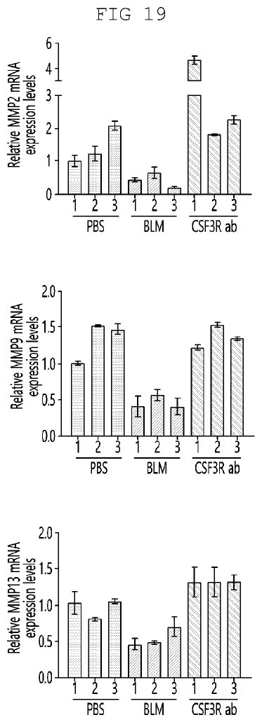

The expression of matrix metalloproteinases (MMPs) related to extracellular matrix (ECM) degradation was analyzed to identify the mechanism that BLM-induced PF-affected lung tissue was restored to normal lung tissue through CSF3R neutralizing antibody treatment. As a result, as shown in FIGS. 18 and 19, the expression of MMP2, MMP9, and MMP13 seeing a BLM-induced decrease thereof was observed to increase again in a mouse model through CSF3R neutralizing antibody treatment. Additionally, when the expression of tissue inhibitors of metalloproteinases (TIMPs), which act as an inhibitor of MMPs, was confirmed, the expression of TIMP-1 and TIMP-2 increased in the BLM-induced pulmonary fibrosis mouse model meanwhile the expression of TIMP-1 and TIMP-2 decreased to the level of normal lung tissue in the group treated with CSF3R neutralizing antibodies.

Through the experiment, it was confirmed in a BLM-induced pulmonary fibrosis mouse model that CSF3R-neutralizing antibodies had a therapeutic effect on pulmonary fibrosis by regulating the expression of MMPs and TIMPs.

### Experiment Example 7. Identification of Downstream Signaling Mechanism Regulating CSF3R-Mediated Epithelial-to-Mesenchymal Transition (EMT)

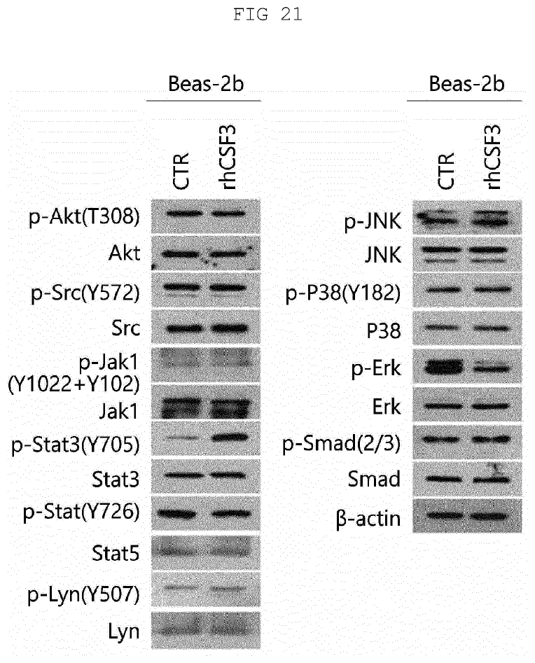

To identify a downstream signaling mechanism that induces epithelial-to-mesenchymal transition (EMT) of lung epithelial cells by CSF3R or CSF3, screening was performed by treating lung epithelial cell line Beas-2b with rh-CSF3. The results are shown in FIG. 21. As shown in FIG. 21, it was observed that among the downstream signaling pathway factors, the activity of STAT3, in particular, increased significantly.

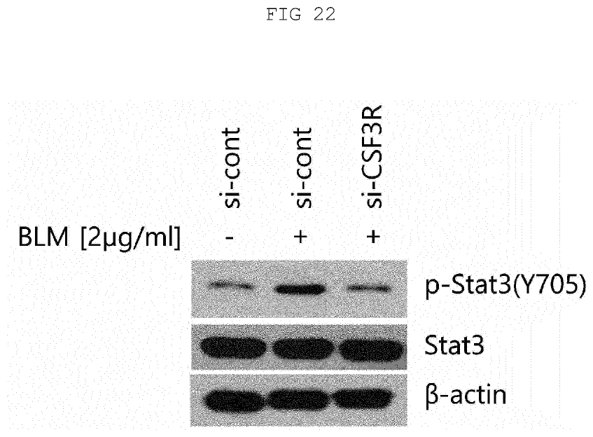

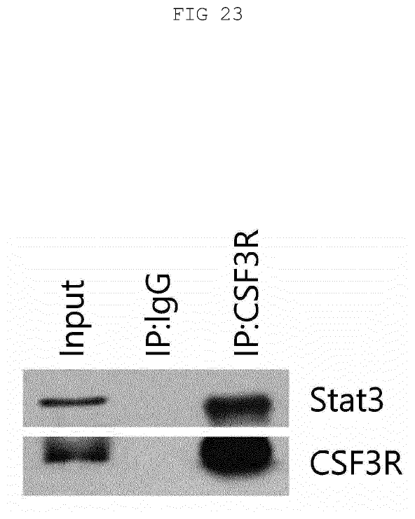

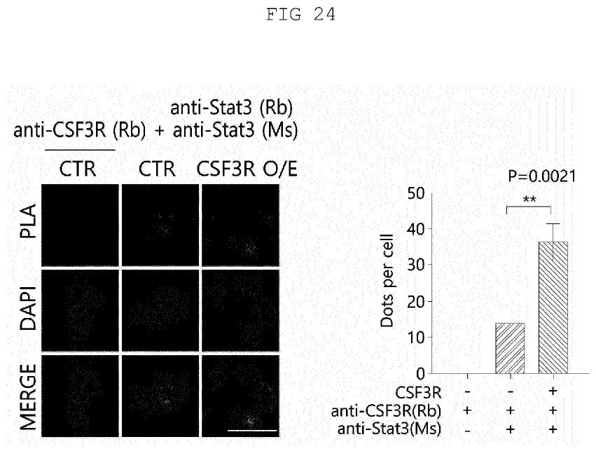

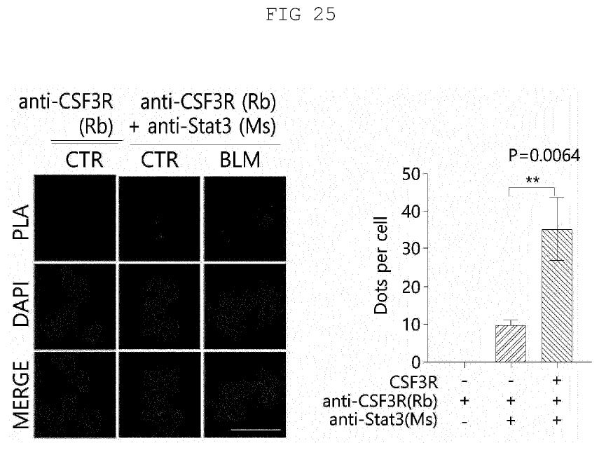

In addition, it was confirmed through western blotting that the BLM-induced activity of STAT3 decreased as the expression of CSF3R was inhibited (see FIG. 22). Direct binding of CSF3R and STAT3 was verified by co-immunoprecipitation (co-IP) (see FIG. 23). In addition, in-situ PLA assay was performed to further verify whether CSF3R and STAT3 bind directly to each other. The results are shown in FIGS. 24 and 25.

Through the experimental results, it was found that STAT3 which had been activated by increased expression of CSF3R induced epithelial-to-mesenchymal transition (EMT) in lung epithelial cell lines.

In this specification, the role of CSF3R in pulmonary fibrosis and the therapeutic mechanism that can treat pulmonary fibrosis by inhibiting the function or expression of CSF3R are disclosed in detail. Furthermore, a therapeutic drug that can treat pulmonary fibrosis is presented. The present disclosure can be used in the industry in various forms, such as a drug for treating pulmonary fibrosis, a pharmaceutical composition containing the same, use of the drug/pharmaceutical composition for treating pulmonary fibrosis, and/or a method of treating pulmonary fibrosis.

1. A method for treating pulmonary fibrosis, comprising:
administering a therapeutically effective amount of anti-CSF3R antibody to a patient in need of treatment,
wherein the anti-CSF3R antibody promotes a degradation of an accumulated extracellular matrix in the lung tissue of the patient.

2. The method of claim 1, wherein the promoting the degradation of the accumulated extracellular matrix comprises reducing (decreasing) an expression level of one or more markers selected from Col1a1, OPN, VER, FN, and Has3.

3. The method of claim 1, wherein the accumulated extracellular matrix is an accumulated collagen.

4. The method of claim 3, wherein the degradation of the accumulated collagen is promoted by at least one of the followings:
an increase (or increasing) of an expression of matrix metalloproteinase (MMP) in the lung tissue of the patient; and
a decrease (or decreasing) of an expression of tissue inhibitors of metalloproteinase (TIMP) in the lung tissue of the patient.

5. The method of claim 4, wherein the matrix metalloproteinase (MMP) is one or more selected from MMP2, MMP9, and MMP13.

6. The method of claim 4, wherein the tissue inhibitors of metalloproteinase (TIMP) is one or more selected from TIMP-1 and TIMP-2.

7. A method for treating pulmonary fibrosis, comprising:
administering a therapeutically effective amount of an anti-CSF3R antibody to a patient in need of treatment,
wherein the anti-CSF3R antibody performs a function selected from the followings, in a lung tissue of the patient:
inhibiting a CSF3R-mediated epithelial to mesenchymal transition (EMT) in a cell within the lung tissue of the patient; and
reverting a cell in the lung tissue of the patient, in which the CSF3R-mediated epithelial to mesenchymal transition (EMT) is occurred, into an epithelial cell or fibroblast.

8. The method of claim 7, wherein the inhibiting of CSF3R-mediated EMT, or the reverting a cell which the CSF3R-mediated EMT is occurred induces one or more selected from the followings:
decreasing an expression of one or more markers selected from Fibronectin, Vimentin, N-cad, and ZEB1; and
increasing an expression of the E-cad.

9. The method of claim 7, wherein the anti-CSF3R antibody is capable of inhibiting or decreasing a binding of STAT3 with CSR3R in a cell of the lung tissue of the patient.

10. The method of any one of claim 1, wherein the pulmonary fibrosis is selected from an idiopathic pulmonary fibrosis, a pulmonary inflammatory fibrosis disease, a chronic obstructive pulmonary disease, and a fibrosis disease occurring in asthma.

11-20. (canceled)

21. The method of claim 7, wherein the pulmonary fibrosis is selected from an idiopathic pulmonary fibrosis, a pulmonary inflammatory fibrosis disease, a chronic obstructive pulmonary disease, and a fibrosis disease occurring in asthma.


SEQUENCE LISTING (1 sequence)

SEQ ID NO: 1 - protein, 836 aa, Homo sapiens
MARLGNCSLTWAALIILLLPGSLEECGHISVSAPIVHLGDPITASCIIKQNCSHLDPEPQILWRLGAELQPGGRQQRLSDGTQESIITLPHLNHTQAFLSCCLNWGNSLQILDQVELRAGYPPAIPHNLSCLMNLTTSSLICQWEPGPETHLPTSFTLKSFKSRGNCQTQGDSILDCVPKDGQSHCCIPRKHLLLYQNMGIWVQAENALGTSMSPQLCLDPMDVVKLEPPMLRTMDPSPEAAPPQAGCLQLCWEPWQPGLHINQKCELRHKPQRGEASWALVGPLPLEALQYELCGLLPATAYTLQIRCIRWPLPGHWSDWSPSLELRTTERAPTVRLDTWWRQRQLDPRTVQLFWKPVPLEEDSGRIQGYVVSWRPSGQAGAILPLCNTTELSCTFHLPSEAQEVALVAYNSAGTSRPTPVVFSESRGPALTRLHAMARDPHSLWVGWEPPNPWPQGYVIEWGLGPPSASNSNKTWRMEQNGRATGFLLKENIRPFQLYEIIVTPLYQDTMGPSQHVYAYSQEMAPSHAPELHLKHIGKTWAQLEWVPEPPELGKSPLTHYTIFWTNAQNQSFSAILNASSRGFVLHGLEPASLYHIHLMAASQAGATNSTVLTLMTLTPEGSELHIILGLFGLLLLLTCLCGTAWLCCSPNRKNPLWPSVPDPAHSSLGSWVPTIMEEDAFQLPGLGTPPITKLTVLEEDEKKPVPWESHNSSETCGLPTLVQTYVLQGDPRAVSTQPQSQSGTSDQVLYGQLLGSPTSPGPGHYLRCDSTQPLLAGLTPSPKSYENLWFQASPLGTLVTPAPSQEDDCVFGPLLNFPLLQGIRVHGMEALGSF